(19) United States
(12) Patent Application Publication
(10) Pub. No.: US 2010/0143462 A1
(43) Pub. Date: Jun. 10, 2010

(54) NEW CARBAMAZEPHINE FORMULATIONS HAVING INPROVED SOLUBILITY

(75) Inventors: Nissim Garti, Jerusalem (IL); Abraham Aserin, Jerusalem (IL); Anna Kogan, Rishon Lezion (IL)

Correspondence Address: BROWDY AND NEIMARK, P.L.L.C.;624 NINTH STREET, NW, SUITE 300, WASHINGTON, DC, 20001-5303, US

(73) Assignee: NUTRALEASE LTD., Ashkelon (IL)

(21) Appl. No.: 11/720,969

(22) PCT Filed: Dec. 6, 2005
(86) PCT No.: PCT/IL2005/001308

§ 371 Date: Feb. 16, 2010

Related U.S. Application Data
(60) Provisional application No. 60/633,127, filed on Dec. 6, 2004.

(51) Int. Cl. A61K 9/66; A61K 31/55

(52) U.S. Cl. 424/455; 514/217

(57) ABSTRACT

The present invention relates to pharmaceutical compositions in the form of microcmulsions comprising carbamazcpinc and their enhanced permeability and extended release properties. The microcmulsion composition may be an oil based formulation or a oil/aqueous phase mixed formulation.

### FIELD OF THE INVENTION

This invention relates to novel oral formulations of Carbamazepine for enhanced bioavailability.

### BACKGROUND OF THE INVENTION

Poor solubility of many orally administered drugs in water leads to restricted bioavailability due to their slow dissolving, poor dispersion and low absorption in the gastro-intestinal tract. By poorly “water soluble drug” is meant a drug that is insoluble in water or has an aqueous solubility of less than about 5 part per 1000 parts of water by weight at 20° C.

Scientists are making many attempts to find new and most efficient vehicles to carry the poorly soluble drugs into our blood stream. These new vehicles should entrap drugs, enhance their solubility and bioavailability and transport via the digestive tract membrane.

Carbamazepine, 5H-Dibenz(b,f)azepine-5-carboxamide (structure given in FIG. 1) has anticonvulsant properties, which have been found useful in the treatment of psychomotor epilepsy and as an adjunct in the treatment of partial epilepsies, when administered in conjunction with other anticonvulsant drugs to prevent the possible generalization of the epileptic discharge. A mild psychotropic effect has been observed in some patients, which seems related to the effect of the Carbamazepine in psychomotor or temporal lobe epilepsy. It is commercially available in the form of tablets, chewable tablets, syrups and extended release formulations. Carbamazepine Carbamazepine relieves or diminishes the pain associated with trigeminal neuralgia often within 24 to 48 hours.

Carbamazepine given as a monotherapy or in combination with lithium or neuroleptics has been found useful in the treatment of acute mania and the prophylactic treatment of bipolar (manic-depressive) disorders.

Carbamazepine is a poorly water-soluble drug (0.11 gr/l at 25° C., i.e. 110 ppm). Pharmacokinetic studies have shown it to be slowly and erratically absorbed from the gastro-intestinal tract when administered in tablet form. Carbamazepine is used for systemic applications, which have many disadvantages such as the need for high dosages (The regular dosage for an adult is 800-1200 mg per day, but in different cases it comes up to 1600 mg), toxicity to the organs like liver and others, side effects at unaffected tissues and long-lasting results. Carbamazepine may cause adverse hematological effects, neuropathy and hypersensitivity syndrome including dermatitis. The enhancement of its solubility leading to higher bioavailability may be crucial in decreasing the dosage and the side effects.

Like other tricyclic compounds, Carbamazepine has a moderate anticholinergic action which is responsible for some of its adverse effects. A tolerance may develop to the action of Carbamazepine after a few months of treatment and should be watched for.

Carbamazepine may suppress ventricular automaticity due to its membrane-depressant effect similar to that of quinidine and procainamide, associated with suppression of phase 4 depolarization of the heart muscle fibre. A number of investigators have reported a deterioration of EEG abnormalities with regard to focal alterations and a higher incidence of records with nil beta activity during Carbamazepine-combined treatment.

When taken in a single oral dose, the Carbamazepine tablets and chewable tablets yield peak plasma concentrations of unchanged Carbamazepine within 4 to 24 hours. With respect to the quantity of Carbamazepine absorbed, there is no clinically relevant difference between the various dosage forms. When the Carbamazepine controlled-release tablets are administered repeatedly, they yield a lower average maximal concentration of Carbamazepine in the plasma, without a reduction in the average minimal concentration: This tends to result in a lower incidence of intermittent concentration-dependent adverse drug reactions. It also ensures that the plasma concentrations remain largely stable throughout the day, thereby making it possible to manage with a twice-daily dosage. Carbamazepine is bound to serum proteins to the extent of 70 to 80%. The concentration of unchanged substance in the saliva reflects the non-protein-bound portion present in the serum (20 to 30%). The elimination half-life of unchanged Carbamazepine in the plasma averages approximately 36 hours following a single oral dose, whereas after repeated administration, which leads to autoinduction of hepatic enzymes, it averages only 16 to 24 hours, depending on the duration of the medication. In patients receiving concomitant treatment with other enzyme-inducing anti-epileptic agents, half-life values averaging 9 to 10 hours have been found. Only 2 to 3% of the dose, whether given singly or repeatedly, is excreted in the urine in unchanged form. The primary metabolite is the pharmacologically active 10,11-epoxide. In man, the main urinary metabolite of Carbamazepine is the trans-diol derivative originating from the 10,11-epoxide; a small portion of the epoxide is converted into 9-hydroxymethyl-10-carbamoyl-acridan. Other important biotransformation products are various monohydroxylated compounds, as well as the N-glucuronide of Carbamazepine. The therapeutic range for the steady-state plasma concentration of Carbamazepine generally lies between 4 and 10 mcg/mL (http://www.mentalhealth.com/drug/p30-t01.html#Head_{—}1).

Carbamazepine can be prepared as described in U.S. Pat. No. 2,948,718. Patent SK279243B describes the preparation of Carbamazepine in one-step reaction of 5-carbamoyl-5H-dibenz[b,f]azepine with a cyanic acid. Other patents U.S. Pat. No. 6,245,908, EP1026158 and U.S. Pat. No. 4,847,374 describe its preparation by using iminostilbene reacted with urea in a protonating medium. It can be administered, e.g. under the trademarks Tegretol™ and Calepsin™.

The mechanism of Carbamazepine action and metabolism are poorly understood. Because of its severe side effects that in some cases may lead to discontinuation of treatment new ways of delivering Carbamazepine with reduced side effects are required.

United states patent US2003/0100884 uses Carbamazepine or its combination with other drugs for enhanced absorption in topical treatment of pain syndromes using iontophoretic treatment. Firstly the preselected neurodermal point for receiving the pharmaceutical agent is located on a subject. Secondly, an ionphoretic patch containing the pharmaceutical agent applied to the subject over the preselected neurodermal point. Thirdly, the delivery of the pharmaceutical agent to the subject at the neurodermal point is done by applying the electrical potential. The Unites States Patent (Provisional application No. 60/068370), Pub No. US2002/0198192A1

reveals other topical formulation comprising a Carbamazepine suspended or dissolved in semisolid vehicle that may be a cream, ointment or gel liquid or lotion that treats the psoriasis disease. The U.S. Pat. No. 6,290,986 and WO9911208 disclose a method for transdermal delivery of Carbamazepine using a matrix of lecithin organogel.

The U.S. Pat. No. RE 34990 and F. Theeuwes in J. Pharm. Sci., (64) 12, 1987-1991, 1975 describe an oral osmotic system. The system comprises of the core containing Carbamazepine, semi permeable protective colloid wall and a passage through the wall. The suspension of the drug is released from the passageway due to the pressure that is built up after permeation of water from body fluids through the semi permeable wall. The osmotic system encountered a problem when the large needles of dihydrate formed in the presence of water from the anhydrous Carbamazepine blocked the aperture of the osmotic system. Using the hydroxypropyl methylcellulose as a protective colloid solved this problem. There are other disadvantages of the osmotic system. The manufacturing of such systems is expensive, signifies a great efforts and environmentally unsafe because it uses coatings based on organic solvents for the semi permeable wall and the passageway using mechanical techniques. The patent U.S. Pat. No. 5,284,662 describes other oral osmotic system comprised of a core with the Carbamazepine, a wall around the core and a bore that connects the core and the environment outside the wall.

The U.S. Pat. No. 5,888,545 discloses an aqueous plasticised polymer dispersion that is applied on Carbamazepine crystals mixed with auxiliarly substances without causing the anhydrous Carbamazepine to convert to dihydrate in the presence of water, that may be filled into capsules or shaped into tablets. Other technique for preventing the formation of dihydrate is described in the patent DE2377520. The Carbamazepine is mixed with inactive tableting ingredients and filled into capsules or pressed to core which are coated with a methacrylic acid-methacrylic acid ester mixture dissolved in isopropranolol.

The U.S. Pat. No. 5,122,543 presents a delayed release formulation comprising Carbamazepine for improved oral administration such as syrups. It contains hydrate crystals of Carbamazepine in cubic or cuboid shapes suitable for stable suspensions and minimum particle size larger then 10 μm and smaller then 200 μm suitable for delayed release and aqueous dispersion where the water soluble polymeric protective colloids such poly-N-vinyl-methylacetamide are suspended. Other adjuvants may be added for oral administration such as sweeteners, anti-oxidants, preservatives, colourings, wetting agents and substances increasing the viscosity.

Since oral administration to an epileptic patient in the emergency situation is not possible parenteral formulation that may be suitable for intravenously administration was divulged in Patent WO 99/18966 comprising of Carbamazepine and a solvent consisting of water and optionally water miscible organic co-solvent with no other solubilizing aids. The immediate response of this formulation is obtained in emergency cases. The accurate and fast active agent dose or blood concentration may be obtained since no adsorption is required. The patent EA4700 divulges other parenteral formulation where more than 90% of the active substance such as Carbamazepine is bound to the applied plasma protein in an aqueous medium in spontaneous equilibrium and room temperature. The EP0435826 and CA2033118 patents relate to a pharmaceutical composition for the intravenous administration of Carbamazepine containing an etherified water-soluble gamma-cyclodextrin derivative as solubiliser. The patent EP0400609 describes other pharmaceutical composition for parenteral use with rapid onset action comprising of Carbamazepine, beta -cyclodextrin etherified by C1-C4-alkyl and/or hydroxy-C2-C4-alkyl as solubiliser. The patent DE4211883 and DE3813015 reveal heat sterizable, stable Carbamazepine solution for parenteral administration that comprise Carbamazepine dissolved in tetrahydrofurfuryl alcohol, polyethylene glycol; ether, water and polyvinyl pyrrolidone. The solution does not form crystals on storage.

The U.S. Pat. No. 5,231,089 discloses makes known a method for improved oral bioavailability of Carbamazepine by complexing it with cyclodextrin selected from the group consisting of hydroxypropyl and hydroxyethyl derivatives of beta—and gamma-cyclodextrin. The WO9517191 describes the use of amino cyclodextrines for the aqueous solubilization of the Carbamazepine. The liquid forms of administration such as syrups or drops have certain advantages over tablets-the dosage can be varied, the absorption is faster due to a rapid dissolution, the uptake is easier so its more suited for children. But the syrups exhibit a disadvantage-due to the presence of fine particles of the active ingredient that dissolve rapidly leading to faster absorption and higher peak plasma levels, the side effects may be increased. This disadvantage does not exist with the tablets.

Microemulsions are the most promising candidates as vehicles for pharmaceutical formulations. The intrinsic physicochemical properties of the microemulsions such as nanometric size, transparency, low viscosity, thermodynamic stability, stability at different pH ranges, ionic environments, thermal stability and high solubilization capacity allow their use in pharmaceutical applications for oral, and inhalation formulations.

The technique of drug solubilization in microemulsions is widely used for enhancing bioavailability of insoluble drugs¹, for protecting proteinic substances from deleterious effects of enzymes², and to targeting of drugs to specific tissues such as lungs³ or tumor cells⁴. Many other potential applications of microemulsions have been studied such as pulmonary⁵, intravaginal or intrarectal administration delivery vehicles for lipophilic drugs such as microcides, steroids, and hormones^{6,7}, and intramuscular formulations of peptide or cell targeting formulations⁸ and other drugs have been also evaluated. There are many additional studies⁹⁻¹² that have shown that ME can help to increase the permeability of the drugs and facilitated transdermic transport.

However, for each drug especially having such low water solubility as Carbamazepine, a specific microemulsion should be elucidated.

### SUMMARY OF THE INVENTION

The present invention is based on the findings that specific microemulsions comprising of a unique blend of components provide effective delivery system for Carbamazepine. Such delivery systems provide enhanced activity with respect to producing an effective amount of Carbamazepine available in the blood.

Thus the invention is directed to a microemulsion pharmaceutical composition comprising:

a. Carbamazepine;

b. An oil phase;

c. at least one C₂-C₅-alcohol as a solvent; and

d. at least one non ionic surfactant.

The relative amounts of the various components are: 0.1-8 wt % carbamazepine, 10-25 wt % oil phase, 10-25 wt % of the at least one C₂-C₅ alcohol as solvent, and 50-70 wt % of said at least one non ionic surfactant.

The pharmaceutical composition may further comprise an aqueous phase which may be water or a mixture of water and an alcohol serving as a co-solvent where the amount of aqueous phase may be up to 95 wt % of the total pharmaceutical composition. The composition may further comprise an amphiphilic co-surfactant. The oil phase is selected from the group consisting of D-limonene, esterified compounds of fatty acids and primary alcohols, propylene glycol mono-C₆₋₁₂ fatty acid esters, glycerol esters of carboxylic acids, medium chain triglycerides having 8 to 20 carbons, in particular 8-14 carbons and most preferred 8-10 carbons, or their mixtures. The C₂-C₅ alcohol is selected from mono hydroxyl alcohols selected from the group consisting of methanol, ethanol, propanol, butanol, pentanol, bi- or tri-hydroxy alcohols selected from the group consisting of ethylene glycol and propylene glycol or their mixtures. The non-ionic surfactant is selected from Brij96, Tween 40, Tween 60 or Tween 80.

The invention is further directed to a method of increasing the bioavailability of carbamazepine in the serum by administering carbamazepine in a composition comprising an oil phase, at least one C₂-C₅-alcohol as a solvent and at least one non ionic surfactant. The composition may further comprise an aqueous phase which may be water or a mixture of water and an alcohol serving as a co-solvent. The composition may further comprise an amphiphilic co-surfactant.

The invention is yet further directed to a method of increasing the permeability of carbamazepine into cells by administering carbamazepine in a composition comprising an oil phase, at least one C₂-C₅-alcohol as a solvent and at least one non ionic surfactant. The composition may further comprise an aqueous phase which may be water or a mixture of water and an alcohol serving as a co-solvent. The composition may further comprise an amphiphilic co-surfactant.

The composition according to the invention may be suited for various forms of administration. It may be administered orally, topically, rectally, vaginally, parenterally, intramuscularly, intradermally, subcutaneously, intraparitoneally, or intravenously. The pharmaceutical system may be in the form of a solution, spray, gel, drops, syrup or elixir, a preconcentrate in a liquid, or as an aqueous or organic diluted preconcentrate. Alternatively it may be in the form of starch capsule, a cellulosic capsule, a hard gelatin capsule or a soft gelatin capsule. It may be formulated for immediate release, controlled release, extended release, delayed release, targeted release, or targeted delayed release.

The invention is further directed to a method of preparing a microemulsion concentrate wherein Carbamazepine is entrapped. The microemulsion may be in an oil based concentrate or an aqueous based concentrate.

### BRIEF DESCRIPTION OF THE DRAWINGS

In order to understand the invention and to see how it may be carried out in practice, a preferred embodiment will now be described, by way of non-limiting example only, with reference to the accompanying drawings, in which:

FIG. 1 shows the chemical structure of Carbamazepine

FIG. 2 shows the solubilization capacity of a microemulsion along dilution line 7:3 according to the present invention containing Carbamazepine, water:propylene glycol (1:1); D-limonene; Tween 60 and ethanol.

FIG. 3 shows the solubilization capacity of a microemulsion along dilution line 8:2 according to the present invention containing Carbamazepine, water:propylene glycol (1:1); D-limonene; Tween 60 and ethanol.

FIG. 4 shows the solubilization capacity of a microemulsion along dilution line 9:1 according to the present invention containing Carbamazepine, water:propylene glycol (1:1); D-limonene; Tween 60 and ethanol.

FIG. 5 shows the solubilization capacity of Carbamazepine along dilution lines 7:3, 8:2, 9:1 normalized to the amount of the oil in the formulation.

FIG. 6 Caco-2 monolayer recoveries in the experimental transport inserts. Transepithelial electrical resistance (TEER) values were measured before (pre-assay) and 24 hours after experiment (post-assay) in the inserts (n=2) of the tested NE-formulations. Results are expressed as Mean TEER values (ohms/cm)±SD. TEER values ≧200 ohm/sec indicate monolayer recovery

FIG. 7 shows permeability coefficients values (Papp) of microemulsions containing Carbamazepine in comparison to Propranolol and Mannitol standards.

FIG. 8 shows a phase diagram of a system comprised of water-PG-D-limonene-EtOH-Tween 60 at 25° C. with constant weight ratio of water:PG (1:1) and a constant weight ratio of D-limonene:EtOH (1:1).

FIG. 9 Caco-2 monolayer recoveries in the experimental transport inserts. Transepithelial electrical resistance (TEER) values were measured before (pre-assay) and 24 hours after experiment (post-assay) in the inserts (n=2) of the tested NE-formulations. Results are expressed as Mean TEER values (ohms/cm)±SD. TEER values ≧200 ohm/sec indicate monolayer recovery

FIG. 10. TEER measurements (%) during transport experiment. Transepithelial electrical resistance (TEER) values were measured at 0, 90 and 180 minutes along time of experiment in inserts (n=2) of tested NE-formulations. Results are expressed as Mean TEER values (in %)±SD.

FIG. 11 Lactate Dehydrogenase (LDH) activities in basolateral buffer of experimental transport inserts for formulations J1high, J1 low. ba1, aa2 and D17. LDH activities in basolateral buffer from inserts of tested formulations (n=2) were measured in a colorimetric assay as described. Results are expressed as optical density (OD) values at 472 nm±SD.

FIG. 12 Lactate Dehydrogenase (LDH) activities in basolateral buffer of experimental transport inserts for formulations A1-A7, A9-A12. LDH activities in basolateral buffer from inserts of tested formulations (n=2) were measured in a colorimetric assay as described. Results are expressed as optical density (OD) values at 472 nm±SD.

FIG. 13 Carbamazepine calculated transport values at Apical to Basolateral direction. Samples from receiver chambers (n=2) were removed at 180 minutes and analyzed in HPLC. Results represent Transport percentage mean values ±SD, calculated as described in methods.

FIG. 14 Carbamazepine calculated transport values at Apical to Basolateral direction. Samples from receiver chambers (n=2) were removed at 180 minutes and analyzed in HPLC. Results represent Transport percentage mean values ±SD, calculated as described in example 22 for formulations aa2, ba1, J1-low, J1-high and D17

FIG. 15 Carbamazepine calculated transport values at Apical to Basolateral direction. Samples from receiver chambers (n=2) were removed at 180 minutes and analyzed in HPLC. Results represent Transport percentage mean values ±SD, calculated as described in example 22 for formulations A1-A12.

FIG. 16 illustrates a comparative example comparing mean serum concentration of carbamazepine in rats where carbamazepine is given either in the commercial formulation (Tergetol®, suspension) or in a microemulsion formulation according to the present invention.

FIG. 17 illustrates a comparative example comparing mean serum concentration of carbamazepine in rats where carbamazepine is given either in the commercial formulation (Tergetol®, suspension) or in a microemulsion formulation according to the present invention after several hours.

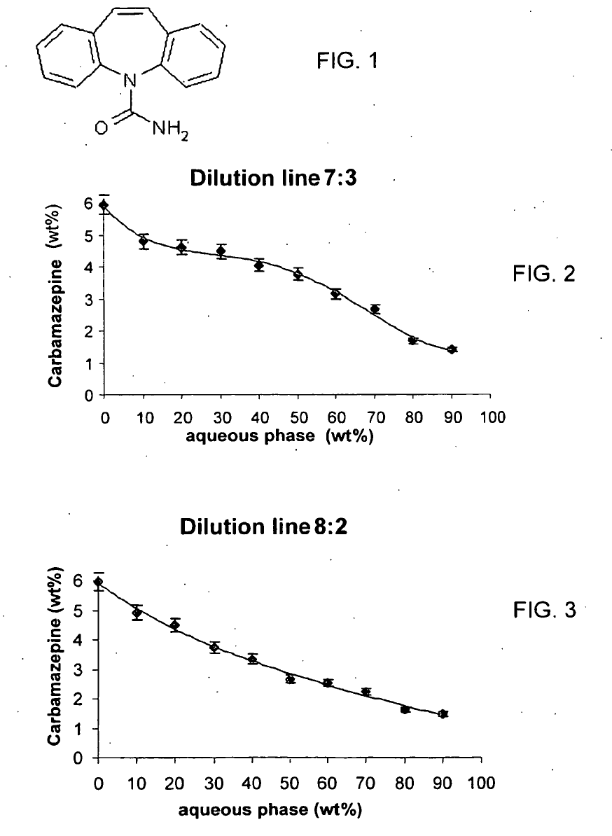

### DETAILED DESCRIPTION OF EXEMPLARY EMBODIMENTS

As mentioned, the invention deals with microemulsion pharmaceutical compositions for Carbamazepine for obtaining an effective delivery of Carbamazepine, attaining an effective blood serum concentration and enhanced permeability of the carbamazepine into cells. The pharmaceutical composition according to the present invention enhances two fold the permeability of Carbamazepine in comparison to the available commercial formulation Tegretol®. The compositions are based on the solubilization of Carbamazepine in a microemulsion. A microemulsion system is a clear, isotropic, thermodynamically stable dispersion of oil, alcohol, surfactant and optionally an aqueous phase which may be water of a water/alcohol mixture. Upon mixture of the oil, alcohol, surfactant and optionally the aqueous phase, thermodynamically stable particles are formed having a diameter of between 8 to 120 nm, preferably 10 to 100 nm. Microemulsion systems contain some definite microstructure, e.g. there is a definite boundary between the oil and water phases at which the surfactant is located. Microemulsions usually contain co-solvents or co-surfactants, which help stabilize the interface, lower the interfacial energy and enable the spontaneous micelle formation. Microemulsions are thermodynamic stable, they are optically clear and easy to prepare. The existence of microdomains of different polarity within the same single-phase solution enables both water-soluble and oil-soluble materials to be solubilized. The surfactant molecules can locate at the oil/water interface. I the present invention non ionic surfactants are employed. Nonionic surfactants, can be used to make either O/W or W/O emulsions. An appropriate surfactant is chosen using the hydrophile-lipophile balance (HLB) score. Surfactants with low HLB values are more lipid loving and thus tend to make a water in oil (W/O) emulsion while those with high HLB values are more hydrophilic and tend to make an oil in water emulsion. The HLB value of each surfactant is determined by an analysis of the characteristics of the surfactant (HLB values for various surfactants are available commercially). It is customary to use a blend of two or more non ionic surfactants rather than a single surfactant molecule. The present invention concerns at least one non ionic surfactant. It may be a blend of several non ionic surfactants, including a hydrophilic non ionic surfactant wherein the overall HLB value of the resulting blend is about 10 and higher. The preferred non ionic surfactants according to the invention are selected from the group consisting of alkylglucosides; alkylmaltosides; alkylthioglucosides; lauryl macrogol glycerides; polyoxyethylene alkyl ethers; polyoxyethylene alkylphenols; polyethylene glycol fatty acids esters; polyethylene glycol glycerol fatty acid esters; polyoxyethylene sorbitan fatty acid esters; polyoxyethylene-polyoxypropylene block copolymers; polyglycerol fatty acid esters; polyoxyethylene glycerides; polyoxyethylene vegetable oils; polyoxyethylene hydrogenated vegetable oils; reaction products of polyols and at least one member of the group consisting of fatty acids, glycerides, vegetable oils, and hydrogenated vegetable oils; sugar esters, sugar ethers; sucroglycerides; and mixtures thereof. The preferred non-ionic hydrophilic surfactant is selected from the group consisting of polyoxyethylene alkylethers; polyethylene glycol fatty acids esters; polyethylene glycol glycerol fatty acid esters; polyoxyethylene sorbitan fatty acid esters; polyoxyethylene-polyoxypropylene block copolymers; polyglycerol fatty acid esters; polyoxyethylene glycerides; polyoxyethylene vegetable oils; polyoxyethylene hydrogenated vegetable oils; reaction products of polyols and at least one member of the group consisting of fatty acids, glycerides, vegetable oils, and hydrogenated vegetable oils; and mixtures thereof.

The non-ionic hydrophilic surfactant may be the reaction product of a polyol and a monoglyceride, diglyceride, triglyceride, or a mixture thereof where the reaction product may comprise a transesterification product. The polyol may be selected from glycerol, ethylene glycol, polyethylene glycol, sorbitol, propylene glycol, pentaerythritol, a saccharide, or a mixture thereof. Hence, the hydrophilic surfactant is selected from the group consisting of PEG-10 laurate, PEG-12 laurate, PEG-20 laurate, PEG-32 laurate, PEG-32 dilaurate, PEG-12 oleate, PEG-15 oleate, PEG-20 oleate, PEG-20 dioleate, PEG-32 oleate, PEG-200 oleate, PEG-400 oleate, PEG-15 stearate, PEG-32 distearate, PEG40 stearate, PEG-100 stearate, PEG-20 dilaurate, PEG-32 dioleate, PEG-20 glyceryl laurate, PEG-30 glyceryl laurate, PEG-20 glyceryl stearate, PEG-20 glyceryl oleate, PEG-30 glyceryl oleate, PEG-30 glyceryl laurate, PEG-40 glyceryl laurate, PEG-40 palm kernel oil, PEG-50 hydrogenated castor oil, PEG-40 castor oil, PEG-35 castor oil, PEG-60 castor oil, PEG-40 hydrogenated castor oil, PEG-60 hydrogenated castor oil, PEG-60 corn oil, PEG-6 caprate/caprylate monoglycerides, PEG-6 caprate/caprylate diglycerides, PEG-8 caprate/caprylate monoglycerides, PEG-8 caprate/caprylate diglycerides, polyglyceryl-10 laurate, PEG-40 sorbitan oleate, PEG-80 sorbitan laurate, polysorbate 20, polysorbate 80, POE-9 lauryl ether, POE-23 lauryl ether, POE-10 oleyl ether, POE-20 oleyl ether, POE-20 stearyl ether, tocopheryl PEG-100 succinate, polyglyceryl-10 oleate, Tween 40, Tween 60, sucrose monostearate, sucrose monolaurate, sucrose monopalmitate, PEG 10-100 nonyl phenol series, PEG 15-100 octyl phenol series, a poloxamer, and combinations thereof. More preferably, the hydrophilic surfactant is selected from the group consisting of PEG-20 laurate, PEG-20 oleate, PEG-35 castor oil, PEG-40 palm kernel oil, PEG-40 hydrogenated astor oil, PEG-60 corn oil, polyglyceryl-10 laurate, PEG-6 caprate/caprylate monoglycerides, caprate/caprylate diglycerides, PEG-8 caprate/caprylate monoglycerides, PEG-8 caprate/caprylate diglycerides, polysorbate 20, polysorbate 80, POE-9 lauryl ether, POE-23 lauryl ether, POE-10 oleyl ether, sucrose monostearate, sucrose monolaurate, a poloxamer, and combinations thereof.

The relationship between the phase behavior of a mixture and its composition may be explained with the aid of a phase diagram. The phase behavior of simple microemulsion systems comprising oil, water and surfactant can be studied with the aid of ternary phase diagram in which each coiner of the diagram represents 100% of that particular component. More commonly, however, and almost always in the case of microemulsions in pharmaceutical applications, the microemulsion will contain additional components such as a cosurfactant and the drug to be microemulsified. The cosurfactant is also amphiphilic with an affinity for both the oil and the aqueous phases and further it partitions in an appreciable extent into the surfactant interfacial monolayer present at the oil-water interface. A wide variety of molecules can function as cosurfactants including non-ionic surfactants and alcohols, alkanoic acids, alkanediols and alkyl amines or their mixtures. Thus the effect of the drug itself on the phase behavior should also be taken into account since a large number of drug molecules are surface active themselves and as such would be expected to influence phase behavior. In a case where the phase behavior of four or more components are investigated, pseudo-ternary phase diagrams are used where a corner will typically represent a binary mixture of two components such as surfactant/cosurfactant, water/drug or oil/drug. The number of different phases present for a particular mixture can be visually assessed. It should be noted that not every combination of components produce microemulsions over the whole range of possible compositions, in some instances the extent of microemulsion formation may be very limited. In most cases, the isotropic regions are separated by two-phase discontinuity regions. Of special interest are U-type microemulsions consisting of single isotropic ‘region with continuous transition, upon dilution, from an oil-rich microemulsion to a water-rich microemulsion without any phase separation (see WO 03/105607). U-type microemulsions for industrial pharmaceuticals and cosmetic applications are based on oils such as hydrocarbons (hexane, dodecane) or fatty acid esters (isopropyl myristate, ethyl laurate, etc.) Similarly, in most cases, the surfactants in use are ionic (AOT, SDS) or non-food-grade ethoxylated fatty alcohols or ethoxylated nonylphenols. The microemulsified drug may be located at one of a number of sites. For example the likely preferred sites of incorporation of a lipophilic, water-insoluble drug into an o/w microemulsion are the disperse oil phase and/or hydrophobic tail region of the surfactant molecule, while a water-soluble material would be most likely to be incorporated in the dispersed aqueous phase of a water-in-oil droplet. The attraction of O/W microemulsion systems lies in their ability to incorporate hydrophobic drugs into the apolar oil phase thereby enhancing their solubility. The dispersal of the drug as a solution in nanometer-sized droplets enhances the rate of dissolution into a contacting aqueous phase, and in vivo generally results in an increase in drug bioavailability. The droplet structure of o/w microemulsions is often retained on dilution by a biological aqueous phase, thereby permitting oral as well as parenteral administration. In contrast, the use of w/o microemulsions for oral or parenteral drug delivery is complicated by the fact that they are destabilized to a much greater extent when diluted by an aqueous phase. This is due to the increase in the volume fraction of the aqueous phase, which increases the ratio of water to surfactant leading to droplet growth and eventually percolation. If the dilution continues, phase separation or inversion may occur and this will result in load dumping.

The microemulsion of the present invention comprises of Carbamazepine, oil, surfactant and ethanol. The formulations of the present invention may further comprise a co-solvent and/or co-surfactant. It may be adjusted for pH and isotonicity as needed, and may also include biocompatable polymers such as protective colloids, building agents and carriers, as needed. Typically, the microemulsion comprises from about 1 to about 6% by weight of Carbamazepine, a much higher capacity than within each solvent only.

In one other preferred aspect, the invention is directed to a microemulsion comprising a) a pharmaceutically effective amount of Carbamazepine, b) an aqueous phase consisted of water and propylene glycol, c) an oil phase, d) an emulsifier selected from the group of the non ionic surfactants (mostly Tween 60 and 80); and e) ethanol. Such a microemulsion diluted in aqueous phase may carry as much as 0.12 wt %. Such an amount being 1200 ppm is more than 10 times the normal solubility of carbamazepine in water (0.11 gr/L. i.e. 110 ppm).

The present invention also contemplates a method of preparing a microemulsion concentrate comprising of the oil phase, the emulsifier, the ethanol and the proper amount of Carbamazepine until the Carbamazepine is completely dissolved, wherein the formulation is a clear liquid at room temperature. The concentrate can further be diluted by an aqueous phase to any dilution needed, e.g. up to 95 wt % water maintaining the microemulsion structure. The diluted system will remain clear. It should hover be noted that the solubilization capacity of the microemulsion depends on the water content (FIGS. 2-4).

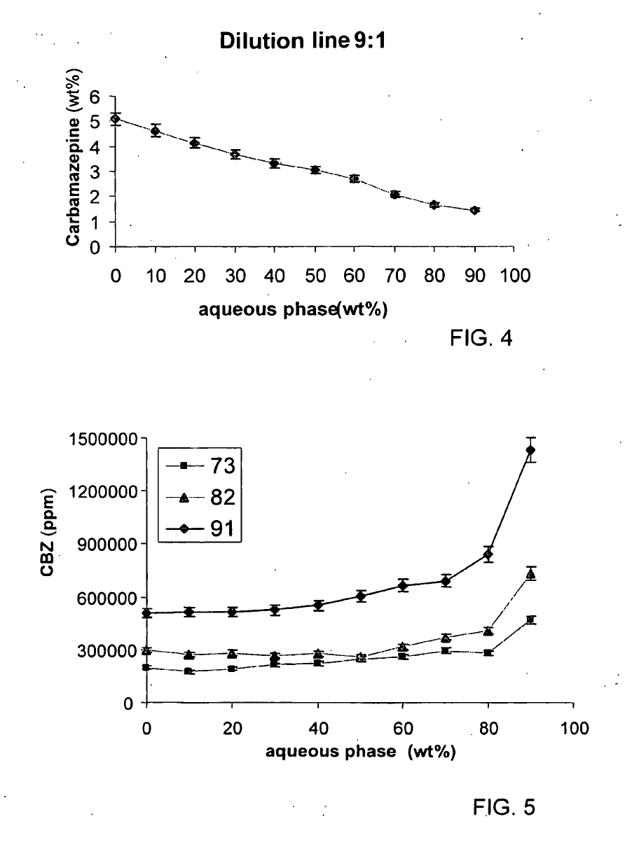

It should be understood that the hydrophobic nature of the active ingredient to be microemulsified, i.e. the Carbamazepine, is a limiting factor at the solubilization process. When solubilizing a drug in a microemulsion system, the solubilization capacity should be characterized. The solubilization capacity of a microemulsion depends on the chemical nature of the critical ingredients at the system, and further on the quantitative relationships between them. FIGS. 2-4 demonstrate the solubilization capacity of a microemulsion according to the present invention containing an aqueous phase and an oil phase along the 7:3, 8:2 and 9:1 dilution lines, respectively where 5 wt % of carbamazepine is solubilized. For the 7:3 dilution line (FIG. 2), the 5 gr oil phase concentrate comprises 0.75 gr of D-limonene, 0.75 gr EtOH (each being 15 wt %), and 3.5 gr Tween 60 (70 wt %). The 5 gr oil concentrate is then diluted in an aqueous phase of water:propylene glycol (1:1). For the 8:2 dilution line (FIG. 3) the 5 gr oil phase concentrate comprises 0.5 gr of D-limonene, 0.5 gr EtOH (each being 10 wt %), and 4 gr Tween 60 (80 wt %). The 5 gr oil concentrate is then diluted in an aqueous phase of water:propylene glycol (1:1). For the 9:1 dilution line (FIG. 4) the 5 gr oil phase concentrate comprises 0.25 gr of D-limonene, 0.25 gr EtOH (each being 5 wt %), and 4 gr Tween 60 (90 wt %). The 5 gr oil concentrate is then diluted in an aqueous phase of water:propylene glycol (1:1).

Another option for a pure oil based formulation is where the oil phase is comprised of D-limonene, ethanol as the solvent and Tween 60 as a surfactant, where the ratio of these three components is 1:1:2.3 (28.8 wt % of the oil and ethanol in a 1:1 ratio and 67.3 wt % of the surfactant with a carbamazepine amount of 3.9 wt %). The aqueous phase, may comprise of only water or a mixture of water and propylene glycol in a 1:1 ratio, where the carbamazepine is again in an initial amount of about 1-3.5 wt %. It is apparent from FIGS. 2-4 that the amount of solubilized drug depends inversely on the aqueous phase content of the microemulsion. The explanation to such a relationship stems from the hydrophobic nature of the drug, as mentioned above. Regardless of the small differences, FIGS. 2-4 are typical demonstrations of microemulsions of the present invention of solubilizing a very hydrophobic material where varying the amount of the aqueous phase in the microemulsion has a big effect on the solubilization capacity.

The benefit of the solubilization of Carbamazepine by the microemulsion system of the present invention may better be understood by normalizing the amount of the solubilized drug versus the amount of the oil in the microemulsion. This parameter will be defined as a (alpha) and will present the solubilized drug amount, in respect to the oil content in the microemulsion as demonstrated in FIG. 5. As apparent from FIG. 5, the normalization reveals that the microemulsion system is able to carry a much higher amount of Carbamazepine, than the amount which could be carried by the oil phase only. This shows in a very clear manner the big advantage of the microemulsion system as a carrier for Carbamazepine apart from the fact that it is an efficient carrier for facile dissolution in the body.

The composition of the present invention, in addition to its ability to solubilize the hydrophobic carbamazepine, further serves as an efficient carrier for carbamazepine which increases the permeability of carbamazepine into cells. Any substance that is absorbed orally must pass the physical barrier of the gastrointestinal epithelium either paracellularly or transcellularly (passive diffusion or carrier mediated). In order to evaluate the efficiency of the microemulsions as efficient carriers for carbamazepine and demonstrate the efficient enhanced permeability of carbamazepine formulated in the microemulsion, a Caco-2 system, based on the intestinal properties of the Caco-2 cells were used. The caco-2 model system represents an in vitro method that is used to predict intestinal permeability of a drug prior to in vivo studies. This way data about potential oral bioavailability is gained. The Caco-2 cells that were used originated from human colon of colorectal carcinoma cells and have similar properties as these of the intestinal epithelium. They preserved the polarity that is seen in the intestinal wall—an apical brush border that faces the intestinal lumen and a basolateral side that faces the body. Cells were grown on membranes, and formed a tight monolayer (width of one cell, as in the intestine) allowing specific compounds to penetrate. Generally, large compounds do not penetrate, but a small compound might penetrate, either in hydrophilic route between the cells (paracellular permeability) or in hydrophobic route (transcellular permeability). Apparent permeability coefficients (Papp) were used to validate the whole experiment. Substances with Papp values of less than 1×10⁻⁶ are considered as having low permeability. Medium permeability substances have Papp values below 1×10⁻⁵. High permeability substances exhibit apparent permeability coefficients in excess of 1×10⁻⁵. Each transport system was conducted using scaling standards and tested substances are compared to these standards. The hydrophilic Mannitol that is low permeability marker is passing via paracellular route. The hydrophobic Propranolol is passing via transcellular route has a high permeability. From FIG. 7 it can be seen that all formulations comprising of microemulsion and Carbamazepine have high Papp values. This high permeability rate indicates transcellular route in accordance with the fact that the drug is hydrophobic.

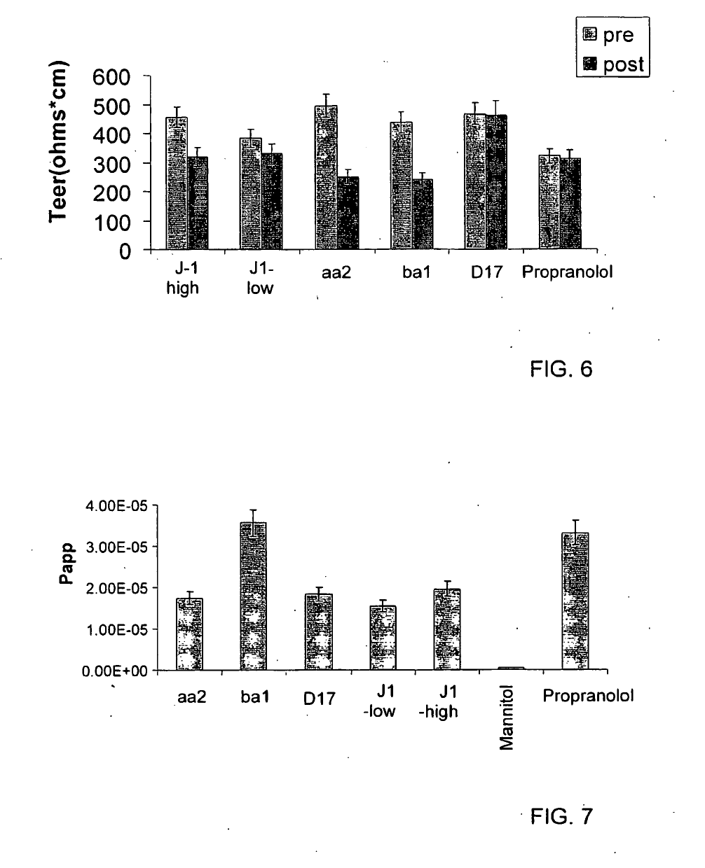

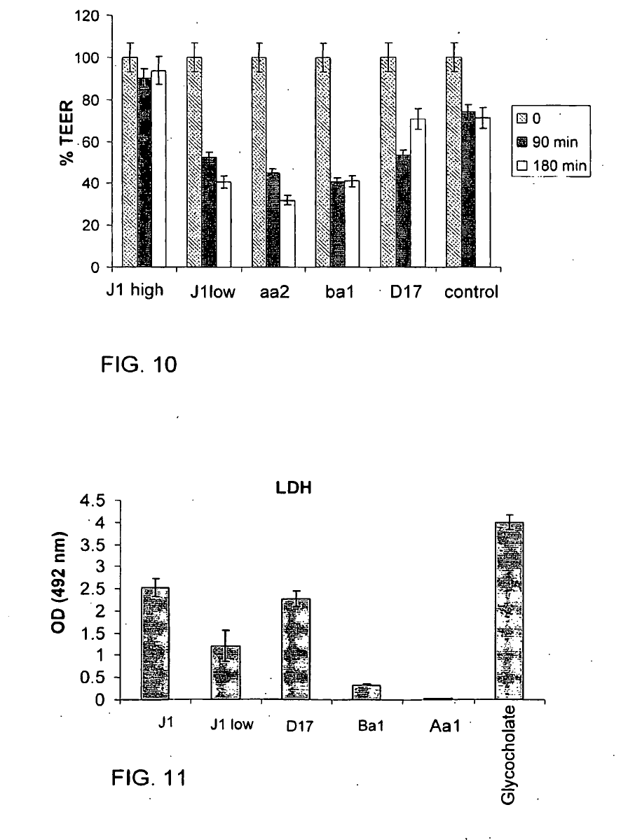

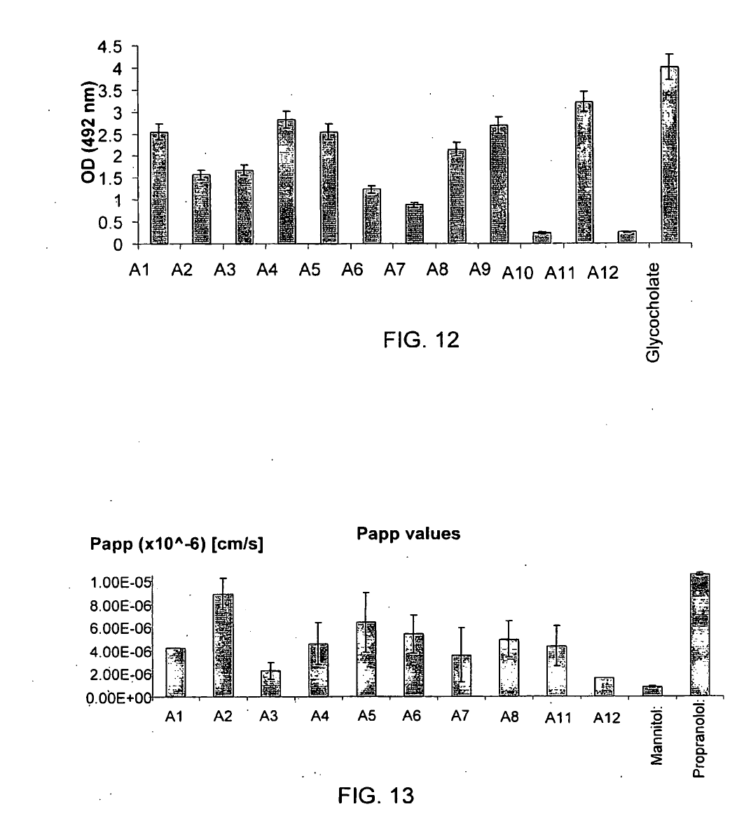

Following the transport experiments, and as prerequisite for any transport analysis, Caco-2 monolayers are assayed in two viability assays to evaluate the effect of the transport of the microemulsion into the cells. Short term damage to cell membrane is examined using LDH release assay (Example and FIGS. 11 and 12), and long term damage is measured by recovery of transepithelial electrical resistance (TEER) values (Example and FIGS. 6 and 9). The enzymatic activity of LDH is measured in a colorimetric assay. A surfactant that damages cell membranes is used as a positive control. Monolayers are considered to recover if their TEER values are ≧200 ohms/cm. Furthermore, the TEER values also demonstrate the enhancement of the microemulsions of the present invention (FIG. 10). Permeability of the Carbamazepine solubilized in microemulsions across the caco-2 monolayers is also shown (FIGS. 13-15). After the transport of the microemulsions, the recovery of the cells must be examined. Since the cells were recovered the transport percentage could be checked and permeability was mechanism evaluated. The microemulsions that were examined did not destroy the Caco-2 cells.

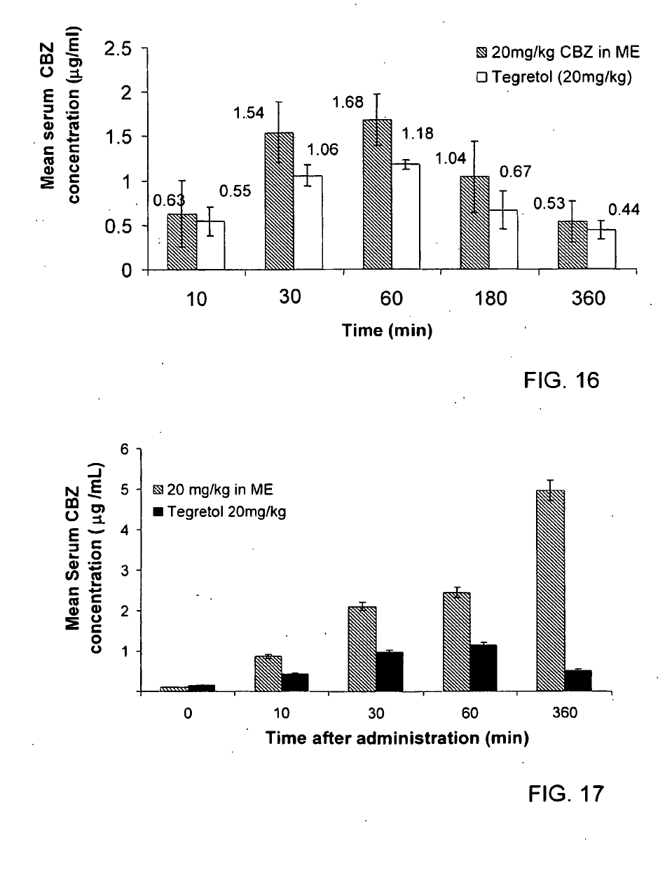

Turning to FIG. 16 there is demonstrated the “time dependent mean serum concentration” of carbamazepine in rats after oral administration of formulations containing carbamazepine in commercial formulation {Tegretol® (100 mg/5 ml)} vs. carbamazepine solubilized in microemulsion of the present invention. Data are shown as the mean concentration, and error bars represent the SD (n=3-6). FIG. 16 clearly demonstrates that at all times intervals, the serum levels of the carbamazepine from the formulation containing 20 mg/kg carbamazepine solubilized in the microemulsion of the preset invention were higher than those measured for the commercial formulation Tegretol. Thus the efficiency of delivering carbamazepine solubilized in the microemulsions of the present invention is higher than that of the commercial Tegretol® suspension. Furthermore, Table I summarizes the Cₘₐₓ, Tₘₐₓ and Area Under Curve (AUC) values for several microemulsion formulations of the present invention compared with the commercial Tegretol® suspension. The Cₘₐₓ values for a 10 mg/kg in a microemulsion is similar to that of 20 mg/kg of Tegretol®. The same equivalence between these two formulations occurs in their AUC values. Hence a lower amount of carbamazepine may be used, yet attain high values of the carbamazepine in the serum. Furthermore, wherein the microemulsion formulations exhibit sharp Tₘₐₓ points (60 minutes or 30 minutes), the Tegretol® formulation varies in its Tₘₐₓ reached between 20 to 60 minutes.

TABLE I

| | | | *AUCtot |
|---|---|---|---|
| | Cₘₐₓ (μg/ml) | Tₘₐₓ | (μg/ml * min) |
| carbamazepine 2 mg\kg in | 0.22 ± 0.03 | 60 ± 0 | 55.70 ± 8.35 |
| microemulaion after 6 hours | | | |
| carbamazepine 10 mg\kg in | 1.54 ± 0.27 | 30 ± 0 | 462.85 ± 50.91 |
| microemulaion after 6 hours | | | |
| carbamazepine 20 mg\kg in | 2.01 ± 0.28 | 60 ± 0 | 630.82 ± 50.46 |
| microemulaion after 6 hours | | | |
| carbamazepine 50 mg\kg in | 2.68 ± 0.32 | 60 ± 0 | 908.14 ± 72.65 |
| microemulaio after 6 hours | | | |
| Tegretol 20 mg/kg | 1.14 ± 0.16 | 40 ± 20 | 468.94 ± 70.34 |
| After 24 hr | | | |
| Tegretol 20 mg/kg | 1.14 ± 0.16 | 40 ± 20 | 449.70 ± 71.49 |
| After 6 hr | | | |

*AUC-area under curve representing the overall amount of carbamazepine administered to the rat.

In yet a separate experiment, an analysis of the serum levels of the solubilized (present invention) and suspended (Tegretol®) carbamazepine reveals in FIG. 17 that whereas the suspended carbamazepine is a short term effect, the maximum serum concentration reaches its maximum between 30 to 60 minutes after oral administration, the solubilized form of the present invention has more constant release where the amount in the serum constantly increases such that after 6 hours the concentration continues growing.

### Examples

General

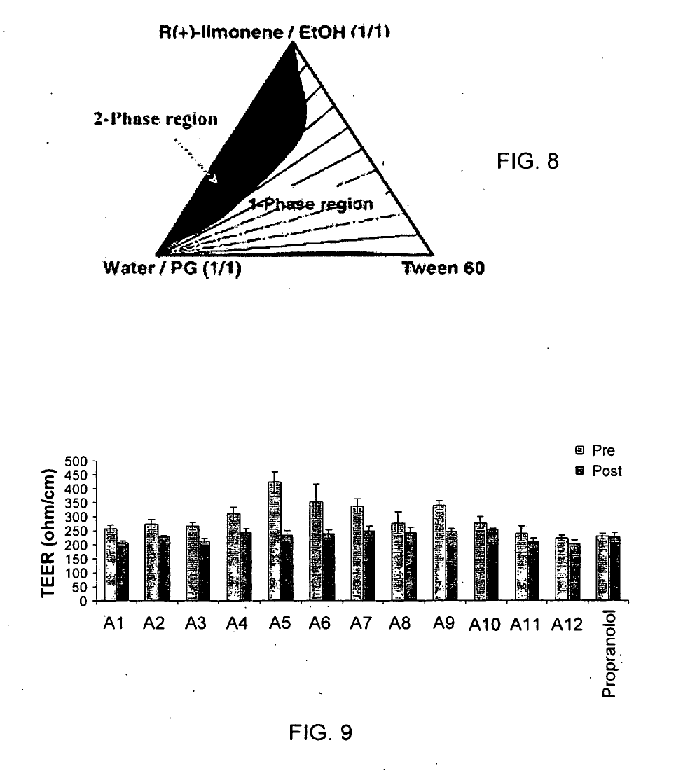

Preparations of the microemulsions and microemulsion concentrate were done in the following manner. The surfactant and the oil phase were mixed at the appropriate ratio, 7:3, 8:2 and 9:1 respectively, to form the concentrate. The solubilization of the drug in the concentrate was determined by solubilizing it to form clear solutions, until the maximal amount was reached. At this case, the drug solubility in the concentrate is approximately 6 wt %. In FIG. 8 there is given a phase diagram of the following system: water-PG-D-limonene-EtOH-Tween 60 at 25 ° C. with constant weight ratio of water:PG (1:1) and a constant weight ratio of D-limonene:EtOH (1:1).

Formulations from other phase diagrams were examined too. The examined systems consisted of water-TriAcetine-Tocopherol Acetate (or Tocopherol)-EtOH-Tween 60 (or Tween 40 or Tween 80) at 25° C. with constant weight of oil phase TriAcetine-Tocopherol Acetate-EtOH (3:1:4), (1:1:2), (2:1:3) at surfactant to oil phase ratio 6:4. In some cases different concentrations of the carbamazepine were solubilized in order to see if amount of the drug influences its transport. The microemulsions containing the drug were prepared by adding the aqueous phase in appropriate proportions, to the “loaded” concentrate and stirring until the solution became homogeneous and clear. In some cases the formulations were heated to 40° C. in order to dissolve the drug in the formulation. Only clear solutions are referred to as microemulsions with solubilized Carbamazepine.

### Example 1

Preparation of the Loaded Microemulsions Composition: A Microemulsion Concentrate Containing 5 wt % of Solubilized Carbamazepine.

The preparation of the concentrate was as mentioned above (general), by mixing 28.8 wt % of a mixture of D-Limonene:ethanol 1:1 with 67.3 wt % of Tween 60, to form a “7:3 concentrate”. In the next step 3.85 wt % of Carbamazepine were solubilized in the concentrate and the solution was stirred. This formulation is slightly yellow colored, clear and stable. As shown at the phase diagram (FIG. 8), this concentrate may be totally diluted by an aqueous phase with no phase separation. Thus such a concentrate may be taken orally where it dilution in the stomach should not form any disintegration of the concentrate upon its dilution. Each 20.8 grams of the composition contain 800 mg Carbamazepine, which is normal dose usually consumed. However, it should be borne in mind that in the present composition the Carbamazepine bioavailability is much higher; hence the consumed dose required for effective action will be much lower and should be determined. Furthermore, it should be mentioned that except for the microemulsion concentrate form, the drug could be consumed at a diluted, ready microemulsion:

### Example 2

Preparation of a Microemulsion Containing Solubilized Carbamazepine and 50 wt % of Aqueous Phase.

The behavior of the concentrate upon dilution with an aqueous phase was characterized and plotted at a solubilization curve, which indicates the microemulsion solubilization capacity at each dilution level. This microemulsion will be prepared according to the solubilization capacity of the microemulsion containing this amount of aqueous phase (FIG. 2). From the solubilization curve it is apparent that at 50 wt % of . aqueous phase, the microemulsion could carry 3.76% of solubilized Carbamazepine. For higher stability and ease of preparation, it was decided to solubilize 3.5 wt % of the drug at this microemulsion. The oil phase contained D-limonene:ethanol 1:1. Concentrate formation: 25 grams of concentrate were prepared at a surfactant:oil phase ratio of 7:3. Drug solubilization: 1.75 grams of Carbamazepine were solubilized in the concentrate which was stirred till homogenous. Aqueous dilution: 25 grams of aqueous phase, containing water:PG at a 1:1 ratio were added to the loaded concentrate and stirred. The solution formed is pale yellow, clear and stable, and is appropriate for oral consumption. Each gram contains 35 mg of the drug. It should be noted that the Carbamazepine bioavailability is much higher, hence the required consumed dose should be re-determined.

### Example 3

Preparation of a Microemulsion Comprising a “Triacetin-Vitamine E Microemulsion” Containing Solubilized Carbamazepine and 90 wt % of Aqueous Phase.

The oil phase contained triacetin:vitamine E:ethanol at a ratio of 3:1:4. Concentrate formation: 5 grams of concentrate were prepared at a surfactant:oil phase ratio of 6:4. Drug solubilization: 77 mg of Carbamazepine were solubilized in the concentrate which was stirred till homogenous. Aqueous dilution: 45 grams of aqueous phase, containing water were added to the loaded concentrate and stirred. The solution formed is clear and stable, and is appropriate for oral consumption. Each gram contains 1.54 mg of the drug. It must be noticed that maximum value of Carbamazepine solubilized in that formulation will be determined and that the Carbamazepine bioavailability is much higher, hence the required consumed dose should be re-determined too. In order to perform the experiment with Caco-2 model the formulation was diluted in apical buffer prior the experiment and the estimated final drug concentration is 0.77 mg/ml (The formulation named aa2 in FIGS. 6, 7, 10, 11 and 14).

### Example 4

Preparation of a Composition Comprising a “Triacetin-Tocopherol Acetate Microemulsion” Containing Solubilized Carbamazepine and 90 wt % of Aqueous Phase.

The oil phase contained triacetin: tocopherol acetate:ethanol at a ratio of 3:1:4. Concentrate formation: 5 grams of concentrate were prepared at a surfactant:oil phase ratio of 6:4. Drug solubilization: 71 mg of Carbamazepine were solubilized in the concentrate which was stirred till homogenous. Aqueous dilution: 45 grams of aqueous phase, containing water were added to the loaded concentrate and stirred. The solution formed is clear and stable, and is appropriate for oral consumption. Each gram contains 1.42 mg of the drug. It must be noticed that maximum value of Carbamazepine solubilized in that formulation will be determined and that the Carbamazepine bioavailability is much higher, hence the required consumed dose should be re-determined too. In order to perform the experiment with Caco-2 model the formulation was diluted in apical buffer prior the experiment and the estimated final drug concentration is 0.71 mg/ml (The formulation named ba1 in FIGS. 6, 7, 10, 11 and 14).

### Example 5

Preparation of a Composition Comprising a Microemulsion Containing Solubilized Carbamazepine and 90 wt % of Aqueous Phase.

(The formulation named D17 in FIGS. 6 and. 7). The oil phase contained D-Limonene:ethanol at a ratio of 1:3. The surfactant phase comprised of Tween 60: PG at ratio 8:2. Concentrate formation: 5 grams of concentrate were prepared at a surfactant:oil phase ratio of 7:3. Drug solubilization: 88 mg of Carbamazepine were solubilized in the concentrate which was stirred till homogenous. Aqueous dilution: 45 grams of aqueous phase, containing water were added to the loaded concentrate and stirred. The solution formed is clear and stable, and is appropriate for oral consumption. Each gram contains 1.76 mg of the drug. It must he noticed that maximum value of Carbamazepine solubilized in that formulation will be determined and that the Carbamazepine bioavailability is much higher, hence the required consumed dose should be re-determined too. In order to perform the experiment with Caco-2 model the formulation was diluted in apical buffer prior the experiment and the estimated final drug concentration is 0.71 mg/ml (The formulation D17 in FIGS. 6, 7, 10, 11 and 14).

### Example 6

Preparation of a Composition of a Microemulsion Containing Solubilized Carbamazepine and 50 wt % of Aqueous Phase (Formulation J1 in FIGS. 6 and 7).

The oil phase contained D-Limonene:ethanol at a ratio of 1:3. The surfactant phase comprised of Tween 60: PG at ratio 8:2. Concentrate formation: 25 grams of concentrate were prepared at a surfactant:oil phase ratio of 7:3. Drug solubilization: 650 mg of Carbamazepine were solubilized in the concentrate which was stirred till homogenous. Aqueous dilution: 25 grams of aqueous phase, containing water were added to the loaded concentrate and stirred. The solution formed is pale yellow, clear and stable, and is appropriate for oral consumption. Each gram contains 13.04 mg of the drug. It must be noticed that maximum value of solubilized Carbamazepine in that formulation will be determined and that the Carbamazepine bioavailability is much higher, hence the required consumed dose should be re-determined too. In order to perform the experiment with Caco-2 model the formulation was diluted in apical buffer prior the experiment and the estimated final drug concentration is 1.36 mg/ml and 0.27 mg/ml (Formulation J1 high and J1 low respectively in FIGS. 6, 7, 10, 11 and 14).

### Example 7

Preparation of a Composition Comprising a “Triacetin-Tocopherol Acetate Microemulsion” Containing Solubilized Carbamazenine and 90 wt % of Aqueous Phase.

The oil phase contained triacetin: tocopherol acetate:ethanol at a ratio of 3:1:4. Concentrate formation: 5 grams of concentrate were prepared at a surfactant (Tween 60):oil phase ratio of 6:4. Drug solubilization: 60 mg of Carbamazepine were solubilized in the concentrate which was stirred till homogenous. Aqueous dilution: 45 grams of aqueous phase, containing water were added to the loaded concentrate and stirred. The solution formed is clear and stable, and is appropriate for oral consumption. Each gram contains 1.2 mg of the drug. It must be noticed that maximum value of Carbamazepine solubilized in that formulation will be determined and that the Carbamazepine bioavailability is much higher, hence the required consumed dose should be re-determined too. In order to perform the experiment with Caco-2 model the formulation was diluted in apical buffer prior the experiment and the estimated final drug concentration is 0.6 mg/ml (Formulation A2 in FIGS. 9, 12, 13, and 15)

### Example 8

Preparation of a Composition Comprising a “Triacetin-Tocopherol Acetate Microemulsion” Containing Solubilized Carbamazepine and 90 wt % of Aqueous Phase

The oil phase contained triacetin: tocopherol acetate:ethanol at a ratio of 3:1:4. Concentrate formation: 5 grams of concentrate were prepared at a surfactant (Tween 60):oil phase ratio of 6:4. Drug solubilization: 46 mg of Carbamazepine were solubilized in the concentrate which was stirred till homogenous. Aqueous dilution: 45 grams of aqueous phase, containing water were added to the loaded concentrate and stirred. The solution formed is clear and stable, and is appropriate for oral consumption. Each gram contains 0.92 mg of the drug. It must be noticed that maximum value of Carbamazepine solubilized in that formulation will be determined and that the Carbamazepine bioavailability is much higher, hence the required consumed dose should be re-determined too. In order to perform the experiment with Caco-2 model the formulation was diluted in apical buffer prior the experiment and the estimated final drug concentration is 0.46 mg/ml (Formulation A3 in FIGS. 9, 12, 13, and 15).

### Example 9

Preparation of a Composition Comprising a “Triacetin-Tocopherol Acetate Microemulsion” Containing Solubilized Carbamazepine and 90 wt % of Aqueous Phase

The oil phase contained triacetin: tocopherol acetate:ethanol at a ratio of 3:1:4. Concentrate formation: 5 grams of concentrate were prepared at a surfactant (Tween 60):oil phase ratio of 6:4. Drug solubilization: 32 mg of Carbamazepine were solubilized in the concentrate which was stirred till homogenous. Aqueous dilution: 45 grams of aqueous phase, containing water were added to the loaded concentrate and stirred. The solution formed is clear and stable, and is appropriate for oral consumption. Each gram contains 0.64 mg of the drug. It must be noticed that maximum value of Carbamazepine solubilized in that formulation will be determined and that the Carbamazepine bioavailability is much higher, hence the required consumed dose should be re-determined too. In order to perform the experiment with Caco-2 model the formulation was diluted in apical buffer prior the experiment and the estimated final drug concentration is 0.32 mg/ml (Formulation A4 in FIGS. 9, 12, 13, and 15).

### Example 10

Preparation of the Composition comprising a “Triacetin-Tocopherol Acetate Microemulsion” Containing Solubilized Carbamazepine and 90 wt % of Aqueous Phase

The oil phase contained triacetin: tocopherol acetate:ethanol at a ratio of 3:1:4. Concentrate formation: 5 grams of concentrate were prepared at a surfactant (Tween 60):oil phase ratio of 6:4. Drug solubilization: 20 mg of Carbamazepine were solubilized in the concentrate which was stirred till homogenous. Aqueous dilution: 45 grams of aqueous phase, containing water were added to the loaded concentrate and stirred. The solution formed is clear and stable, and is appropriate for oral consumption. Each gram contains 0.4 mg of the drug. It must be noticed that maximum value of Carbamazepine solubilized in that formulation will be determined and that the Carbamazepine bioavailability is much higher, hence the required consumed dose should be re-determined too. In order to perform the experiment with Caco-2 model the formulation was diluted in apical buffer prior the experiment and the estimated final drug concentration is 0.2 mg/ml (Formulation AS in FIGS. 9, 12, 13, and 15).

### Example 11

Preparation of ae Composition Comprising a “Triacetin-Tocopherol Acetate Microemulsion” Containing Solubilized Carbamazepine and 90 wt % of Aqueous Phase

The oil phase contained triacetin: tocopherol acetate:ethanol at a ratio of 3:1:4. Concentrate formation: 5 grams of concentrate were prepared at a surfactant (Tween 40):oil phase ratio of 6:4. Drug solubilization: 60 mg of Carbamazepine were solubilized in the concentrate which was stirred till homogenous. Aqueous dilution: 45 grams of aqueous phase, containing water were added to the loaded concentrate and stirred. The solution formed is clear and stable, and is appropriate for oral consumption. Each gram contains 1.2 mg of the drug. It must be noticed that maximum value of Carbamazepine solubilized in that formulation will be determined and that the Carbamazepine bioavailability is much higher, hence the required: consumed dose should be re-determined too. In order to perform the experiment with Caco-2 model the formulation was diluted in apical buffer prior the experiment and the estimated final drug concentration is 0.6 mg/ml (Formulation A6 in FIGS. 9, 12, 13, and 15)

### Example 12

Preparation of a Composition Comprising a “Triacetin-Tocopherol Acetate Microemulsion” Containing Solubilized Carbamazepine and 90 wt % of Aqueous Phase

The oil phase contained triacetin: tocopherol acetate:ethanol at a ratio of 3:1:4. Concentrate formation: 5 grams of concentrate were prepared at a surfactant (Tween 80):oil phase ratio of 6:4. Drug solubilization: 60 mg of Carbamazepine were solubilized in the concentrate which was stirred till homogenous. Aqueous dilution: 45 grams of aqueous phase, containing water were added to the loaded concentrate and stirred. The solution formed is clear and stable, and is appropriate for oral consumption. Each gram contains 1.2 mg of the drug. It must be noticed that maximum value of Carbamazepine solubilized in that formulation will be determined and that the Carbamazepine bioavailability is much higher, hence the required consumed dose should be re-determined too. In order to perform the experiment with Caco-2 model the formulation was diluted in apical buffer prior the experiment and the estimated final drug concentration is 0.6 mg/ml (Formulation A7 in FIGS. 9, 12, 13, and 15)

### Example 13

Preparation of a Composition Comprising “Triacetin-Tocopherol Microemulsion” Containing Solubilized Carbamazepine and 90 wt % of Aqueous Phase

The oil phase contained triacetin: tocopherol :ethanol at a ratio of 3:1:4. Concentrate formation: 5 grams of concentrate were prepared at a surfactant (Tween 60):oil phase ratio of 6:4. Drug solubilization: 60 mg of Carbamazepine were solubilized in the concentrate which was stirred till homogenous. Aqueous dilution: 45 grams of aqueous phase, containing water were added to the loaded concentrate and stirred. The solution formed is clear and stable, and is appropriate for oral consumption. Each gram contains 1.2 mg of the drug. It must be noticed that maximum value of Carbamazepine solubilized in that formulation will be determined and that the Carbamazepine bioavailability is much higher, hence the required consumed dose should be re-determined too. In order to perform the experiment with Caco-2 model the formulation was diluted in apical buffer prior the experiment and the estimated final drug concentration is 0.6 mg/ml (Formulation A8 in FIGS. 9, 12, 13, and 15)

### Example 14

Preparation of a Composition Comprising a “Triacetin-Tocopherol Acetate Microemulsion” Containing Solubilized Carbamazepine and 90 wt % of Aqueous Phase

The oil phase contained triacetin: tocopherol acetate:ethanol at a ratio of 3:1:4. Concentrate formation: 5 grams of concentrate were prepared at a surfactant (Tween 60):oil phase ratio of 7:3. Drug solubilization: 60 mg of Carbamazepine were solubilized in the concentrate which was stirred till homogenous. Aqueous dilution: 45 grams of aqueous phase, containing water were added to the loaded concentrate and stirred. The solution formed is clear and stable, and is appropriate for oral consumption. Each gram contains 1.2 mg of the drug. It must be noticed that maximum value of Carbamazepine solubilized in that formulation will be determined and that the Carbamazepine bioavailability is much higher, hence the required consumed dose should be re-determined too. In order to perform the experiment with Caco-2 model the formulation was diluted in apical buffer prior the experiment and the estimated final drug concentration is 0.6 mg/ml (Formulation A9 in FIGS. 9, 12, 13, and 15)

### Example 15

Preparation of a Composition Comprising a “Triacetin-Tocopherol Acetate Microemulsion” Containing Solubilized Carbamazepine and 90 wt % of Aqueous Phase

The oil phase contained triacetin: tocopherol acetate:ethanol at a ratio of 1:1:2. Concentrate formation: 5 grams of concentrate were prepared at a surfactant (Tween 60):oil phase ratio of 6:4. Drug solubilization: 60 mg of Carbamazepine were solubilized in the concentrate which was stirred till homogenous. Aqueous dilution: 45 grams of aqueous phase, containing water were added to the loaded concentrate and stirred. The solution formed is clear and stable, and is appropriate for oral consumption. Each gram contains 1.2 mg of the drug. It must be noticed that maximum value of Carbamazepine solubilized in that formulation will be determined and that the Carbamazepine bioavailability is much higher, hence the required consumed dose should be re-determined too. In order to perform the experiment with Caco-2 model the formulation was diluted in apical buffer prior the experiment and the estimated final drug concentration is 0.6 mg/ml (Formulation A10 FIGS. 9, 12, 13, and 15)

### Example 16

Preparation of a Composition Comprising a “Triacetin-Tocopherol Acetate Microemulsion” Containing Solubilized Carbamazepine and 90 wt % of Aqueous Phase

The oil phase contained triacetin: tocopherol acetate :ethanol at a ratio of 2:1:3. Concentrate formation: 5 grams of concentrate were prepared at a surfactant (Tween 60):oil phase ratio of 6:4. Drug solubilization: 60 mg of Carbamazepine were solubilized in the concentrate which was stirred till homogenous. Aqueous dilution: 45 grams of aqueous phase, containing water were added to the loaded concentrate and stirred. The solution formed is clear and stable, and is appropriate for oral consumption. Each gram contains 1.2 mg of the drug. It must be noticed that maximum value of Carbamazepine solubilized in that formulation will be determined and that the Carbamazepine bioavailability is much higher, hence the required consumed dose should be re-determined too. In order to perform the experiment with Caco-2 model the formulation was diluted in apical buffer prior the experiment and the estimated final drug concentration is 0.6 mg/ml (Formulation A11 in FIGS. 9, 12, 13, and 15)

### Example 17

Preparation of a Composition Comprising a “Triacetin-Microemulsion” Containing Solubilized Carbamazepine and 90 wt % of Aqueous Phase

The oil phase contained triacetin. Concentrate formation: 5 grams of concentrate were prepared at a surfactant (d-Alpha-Tocopheryl Polyethylene Glycol-1000 Succinate):oil phase ratio of 6:4. Drug solubilization: 60 mg of Carbamazepine were solubilized in the concentrate which was stirred till homogenous. Aqueous dilution: 45 grams of aqueous phase, containing water were added to the loaded concentrate and stirred. The solution formed is clear and stable, and is appropriate for oral consumption. Each gram contains 1.2 mg of the drug. It must be noticed that maximum value of Carbamazepine solubilized in that formulation will be determined and that the Carbamazepine bioavailability is much higher, hence the required consumed dose should be re-determined too. In order to perform the experiment with Caco-2 model the formulation was diluted in apical buffer prior the experiment and the estimated final drug concentration is 0.6 mg/ml (Formulation A12 in FIGS. 9, 12, 13, and 15)

### Example 18

### Membrane Transport Studies for Formulations Described in Examples 3-18

### Experimental

### Cell Lines

Caco-2 cells, originating from a human colorectal carcinoma, were provided by ATCC (American Type Culture Collection). All cells used in this study were between 50 and 60 passage number.

### Standards

[³H]-Mannitol Standard solution was prepared by dilution of 26.3 Ci/mmol [³H]-Mannitol stock solution to 5.26 Ci/mmol final concentrations in PBS.

Propranolol Standard solution was prepared by diluting the 5 mM stock solution to 1 mM final concentration in PBS.

Commercially available tablet of Carbamazepine (Tegretol) was dissolved in PBS to 1.2 mg/ml, sonicated for 10 minutes at room temparaturetemperature and than centrifuged at 10,000 g for additional 10 minutes. The supernatant was taken to the transport assay. (formulation A1 in FIGS. 9, 12, 13 and 15)

### Buffers and Growth Media

PBS: Dulbeco's Phosphate Buffered Saline without calcium and without magnesium (Biological Industries, Cat. 02-023-1A)

Growth medium: Caco-2 cells were grown in high D-glucose DMEM supplemented with 1% L-glutamine, 1% nonessential amino acids, 1% sodium pyruvate, 1% penicillin-streptomycin and 10% fetal bovine serum.

Cell Culture

Caco-2 cells were cultured in 75 cm² culture flasks. The flasks were kept at 37° C. in an atmosphere of 5% CO₂ and 100% humidity. The culture medium was changed every other day and the day before the experiment. For subculturing, the medium was removed and the cells were detached from the culture flasks with 0.25% Trypsin-EDTA. Culture medium with fetal bovine serum (FBS) was added to stop trypsinization.

Cells (passage 62) were harvested after 95% confluency and seeded at a density of 85,000 cells per polycarbonate membranes (0.4 μm pore size and a surface area of 0.31 cm²) inserts. Cells on inserts were cultured for 7 days.

HPLC Analysis

### Carbamazepine Analytical Parameters

Carbamazepine was detected using WATERS 2790 HPLC, equipped with photodiode array (PDA, Waters 996) at 285 nm.

Column:Merck RP-18 (LichroCART®) 100, 5 μM) at 45° C.

Mobile Phase:

Solvent A: 40%—0.1% H₂SO₄ in DDW (pH adjusted to 3 with NaOH 1M)

Solvent B: 30%—MeOH

Solvent C: 30%—ACN

Flow rate: 1 ml/min.

The Carbamazepine peak elutes at 4.0 minutes.

### Propranolol Standard Parameters

Propranolol was detected using WATERS 2790 HPLC, equipped with photodiode array (PDA, Waters 996).

Column:Waters ODS-3, (Spherisorb® 250 mm, 4.6 mm internal diameter, 5 μm particles diameter) at 35° C.

Mobile phase: Solvent A: Water 0.1% TFA

Solvent B: ACN 0.1% TFA

Gradient elution: 1 ml/min flow rate as described in the table:

Time (minutes)

| | 15 | 20 | 25 | 26 |
|---|---|---|---|---|
| % A | 50 | 5 | 5 | 95 |
| % B | 50 | 95 | 95 | 5 |

### Example 19

Confluency and differentiation of the cell monolayer on inserts was measured by the evaluation of the Trans-Epithelial-Electrical-Resistance (TEER). Monolayers exhibiting similar TEER values consistent with “non leakiness” were used to study and compare transport characteristics of model actives in plain buffer and in presence of diluted compositions of the present invention.

The recovery of the Caco-2 monolayer examined by TEER (transepithelial resistance). It exists across the monolayer because the tight junctions restrict the movement of ions. If tight junction opens the ion flow through the tight junctions is increased and the TEER values are reduced. Monolayers are considered recovered if TEER values are more or equal to 200 ohm/sec. TEER values of the monolayers that are above 200 ohm/sec are considered as recovered. From FIG. 6 and FIG. 9 can be seen that the TEER values measurements in all tested groups showed full monolayers recovery (values are ≧200 ohms/cm), similarly to control. Hence, according to this assay, the pharmaceutical compositions of the present invention in the form of microemulsions did not cause any monolayer damage. The measurement of TEER along experimental time enables to examine enhancer activity of micro emulsion formulations. In order to define a substance as enhancer, the TEER values during experiment should be reduced to 70% below the measured TEER value at time point 0. If 50%-70% reduction in TEER values is measured, then substance is considered as potential enhancer. When TEER values are reduced up to 50% of TEER at time 0 the substance is not considered as potential enhancer. FIG. 10 shows TEER measurements at three time points along transport experiment. Most of the namo liquid-formulations showed the trend of TEER reduction values relative to the values measured at the assay start point (time 0). Since all the-formulations groups monolayers recovered the reduction may be explained as a direct effect of nano vehicle-formulations on the tight junctions that cause the reduction in the monolayers electrical resistance.

According to the data in FIG. 10 the following formulations can be considered as potential enhancers: j1-low, aa2, and ba1 since during the experiment the measured TEER values of these groups were reduced to levels of 50% to 70% relative to start point measurements (time 0).

### Example 20

LDH Assay

LDH assay is an indication of short term damage to the Caco-2 monolayers monolayers.

Following transport experiments, a release of lactate dehydrogenase (LDH) enzyme to the surrounding buffer is examined. The larger the release the larger is the damage to the cells. The enzymatic activity of LDH is measured in a colorimetric assay. A surfactant that damages cell membranes is used as a positive control (Glycocholate). 100 μl of cells supernatant were removed from the basolateral chamber at the end of transport experiment. LDH release was assayed according to the manufacturer instruction using cytotoxicity detection kit (Roche, Cat. No. 1 644 793).

The results illustrated in FIG. 11 show that j1-high, j1-low and d17 formulations were relatively cytotoxic for the cell membranes as compared to the positive control substance (20 mM Glycocholate). Consequently one may observe that formulations ba1 and aa7 were hardly damaging to Caco-2 cells.

The results illustrated in FIG. 12 show that A1, A4, A5, A9, A11 formulations were relatively cytotoxic for the cell membranes as compared to the positive control substance (20 mM Glycocholate). Consequently, formulations A2, A3, A6, A7,A8 and A12 were hardly damaging to Caco-2 cells. Furthermore, FIG. 12 indicates that since formulations A2, A3, A6, A7, A10 and A12 all have has smaller OD values than the commercial formulation A1 the short term damage done to the cells by our formulations is smaller.

### Example 21

Caco-2 monolayer, which serves as GI transport screening system, is a biological system. As such, each specific transport experiment differs from another by the exact cells passage, biological additives, and numerous unknown factors, that might influence the exact performance. To overcome this obstacle, each transport system is conducted using scaling standards, and tested substances are compared to these standards¹. In addition, Pₐₚₚ values of the scaling standards validate the whole experiment. ¹Waiver for In Vivo Bioavailability & Bioequivalence Studies for Immediate-Release Solid Oral Dosage Forms Based on the Biopharmaceutics Classification System, Guidance for Industry, U.S. Department of Health and Human Services, FDA, CDER, August 2000,BP.

Generally, substances with an apparent permeability coefficient (Pₐₚₚ) off less than 1×10⁻⁶ cm/s are classified as low permeability substances, medium-permeability substances have Pₐₚₚ values below 1×10⁻⁵ cm/s and high permeability substances exhibit apparent permeability coefficients in excess of 1×10⁻⁵ cm/s.

### Pₐₚₚ Calculations

Apparent permeability coefficients (Pₐₚₚ) of tested compounds and standards were calculated from the following equation:

Pₐₚₚ₍ₜ₎=(CₜV/t)*(1/C₀A) Equation 1

In which C0 is the initial concentration (at t=0) of the compound on the donor (apical side) and Ct is the concentration at the calculated time point (t) in the receiver chamber, V is receiver chamber volume, A is the surface area of the monolayer, and t is the elapsed time. In kinetic studies, presented Pₐₚₚ is calculated using the slope value.

FIG. 7 and FIG. 13 depict quality grading that was performed using the low permeability marker, Mannitol, (with expected Pₐₚₚ of ≦1×10⁻⁶ cm/s) and the high permeability marker, Propranolol (with expected Pₐₚₚ of at least X5 of Mannitol). From FIG. 7 it can be seen that formulations aa2, ba1, J1-low and J1-high have Papp. coefficients higher then 1×10⁻⁵ cm/s indicating that they are high permeability substances. FIG. 13 shows that formulation A2 exhibits high permeability, A3 and A12 exhibit low-medium permeability, formulation A1, A4-A11 exhibit medium high permeability. From this figure it can be seen that formulations A2, A4, A5, A6, A8 and A11 have larger permeability coefficients then the commercial formulation A1

### Example 22

### Transport Studies

Test substances transport across Caco-2 monolayers was investigated in ‘apical to basolateral’ direction, at one time point (180 minutes for the first study and 120 minutes for the second study), or at several time points (for kinetics study).

Prior to the experiment, the culture medium was removed; cells were washed with PBS and incubated for 30 min at 37° C., 45 RPM.

Transport studies were performed at 37° C., with shaking at 45 RPM with either test substances (n=3) or standards (n=2). If Transport kinetics was conducted, during treatment aliquots were removed form the basolateral chamber at 10, 20, 40, 60, 90, and 120 minutes, and replaced by fresh PBS buffer. At endpoint (120 minutes), the whole volume of the apical and basolateral chambers was collected from all the inserts in the study. For the radioactive marker Mannitol, 20 μl duplicate aliquots were drawn at each time point.

### % Transport Calculation

At the last time point of the experiment (120 minutes), basolateral amounts values were measured and transport percentage for each compound was calculated according to the following equation:

% Transport=100*(C_{120 min}*V_{basolateral})/C₀*V_{apical} Equation 2

In which C_{120 min} is the concentration after 120 minutes of the compound on the receiver (basolateral side), V_{basolateral} is receiver chamber volume, C₀ is the initial concentration (at t=0) of the compound on the donor (apical side) and V_{apical} is donor chamber volume.

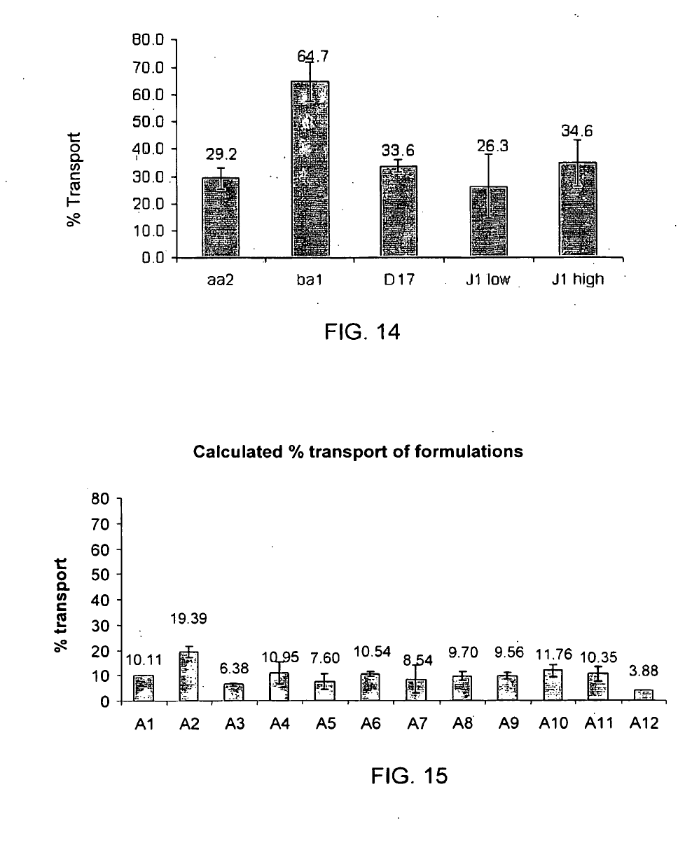

As depicted from FIG. 14 it can be seen that all formulations allowed Carbamazepine transport with values above 25%, as compared to the initial concentrations. All formulations showed similar transport values except for ba1 formulation, where Carbamazepine transport was significantly higher (64.7%). Hence, transport calculations indicate that ba1 formulation may enhance Carbamazepine transport across Caco-2 monolayer. FIG. 15 illustrates that the various formulations exhibit different transport values. Most of the formulations have the same values of transport as the commercial formulation A1 except for the A2 formulation where Carbamazepin's transport is twice higher.

### Cells Recovery

At the end of the experiment, PBS was replaced by growth medium. Then, TEER values were measured and recorded as post TEER values. Inserts were incubated for additional 24 and/or 48 hours for recovery TEER measurements. Inserts with TEER values >200 ohms/cm were considered as recovered.

### Example 23

### A Triacetin-Tocopherol Acetate Microemulsion Containing Solubilized Carbamazepine Compositions for Determining Blood Concentration of Carbamazepine in Rats.

The oil phase contained triacetin: tocopherol acetate:ethanol at a ratio of 3:1:4. Concentrate formation: 5 grams of concentrate were prepared at a surfactant (Tween 80):oil phase ratio of 6:4 (67 wt % Tween 60, 33 wt % oil phase and ethanol where the ethanol is 16.5 wt % and oil phase comprised of a mixture of two oils in a 3:1 ratio is 16.5 wt %). Drug solubilization: 10 mg, 50 mg, 100 mg or 250 mg of Carbamazepine were solubilized in the concentrate (o.2 wt % to 5 wt %) which was stirred till homogenous. These concentrations are corresponding to doses of 2 mg/kg, 10 mg/kg, 20 mg/kg, 50 mg/kg of rat. Tegretol® (100 mg/5 ml) is the commercial oral suspension of carbamazepine and was used immediately after purchased.

In vivo studies [This study was approved by the faculty ethics committee (No. MD 106.20-2) and has NIH approval (No. OPRR A01-5011)].

The rat model is widely used as an animal model of drug bioavailability. The pharmacokinetic study of carbamazepine was conducted in fed male Sprague-Dawley rats (Harlan, Israel) weighing 200±50 g. Rats were housed under clean conventional conditions and handled for a 1-week period to allow them to acclimatize. Each rat was identified by tail mark and cage number. The right or left jugular vein of the rats was cannulated with polyethylene tubing (PE-50) under anesthesia of Ketamin-xylazine anesthesia (50 mg/kg and 2 mg/kg IP.) the day before the pharmacokinetics experiment and up to 2 weeks from for the following experiments. Rats were given the formulation by oral gavage using a special gavage needle and blood was taken from the cannulation tube in the following timepoints: 0 min, 5 min, 10 min, 15 min, 30 min, 1 h, 2 h, 3 h, 6 h 9 h, 12 h, 24 after dosing (depending on the formulation).

Blood samples (0.25 ml) were collected through the cannula in heparinized microcentrifuge plastic tubes before several time points after dosing. Each collected blood sample was centrifuged at 5000 g for 10 min at 8-10° C. The plasma fraction was frozen at −20° C. until analysis, and the FIPA assays for analyzing the CBZ concentrations in collected blood were performed within 1 week of collection. After the end of the experiment each rat received 3 ml LRS (Hartman's solution) via the central catheter in order to maintain proper blood volume. The central cannulae were then washed with Heparin-glycerol to maintain open cannules for future experiments. Three to six animals were used for each formulation.

### Cannulation of the Jugular Vein

For surgical anesthesia, a mixture of Ketamine-Xylazine was administered IP and the surgical area was trimmed and prepared aseptically.

A 1 to 1.5 cm skin incision is made over the ventral thorax slightly to the left of center. The jugular vein passes under the right clavicle into the chest cavity. A portion of the vein was freed from all underlying tissue. After isolating an approximately 5 mm section of the vein; two fine silk ligatures are placed at either end of the isolated portion of the vein. The ligature closest to the head was tied to occlude blood flow going to the heart. With a pair of small hemostats or locking forceps, we grasped the ends of the tied ligature and positioned the hemostats to exert slight tension towards the head.

A venotomy was performed in the isolated portion of the vein close to the tied cranial ligature with the point of a 25 ga. needle. We inserted the closed tips of fine Dumont forceps into the venotomy and allowed the tips to open slightly to distend the opening. We Inserted the polyethylene tubing (PE-50) into the lumen of the vein using the opening of the Dumont forceps as a guide. We advanced the tube to the untied ligature and inserted the catheter until the retention area of the catheter is over the venotomy site. We tied the ligature closest to the clavicle around the jugular vein and catheter and the ligature closest to the head around the catheter. One end of each ligature was tied together. The patient was turned over and small skin incision was made between the scapulae. A subcutaneous tunnel was made from this incision to the incision over the right thorax. The catheter port was grasped pulled through the incision between the scapulae. The scapular incision and the ventral skin incision was closed.

Turning to FIG. 16 there is demonstrated the “time dependent mean serum concentration” of carbamazepine in rats after oral administration of formulations containing carbamazepine in commercial formulation {Tegretol® (100 mg/5 ml)} vs. carbamazepine solubilized in microemulsion of the present invention. Data are shown as the mean concentration, and error bars represent the SD (n=3-6). FIG. 16 clearly demonstrates that at all times intervals, the serum levels of the carbamazepine from the formulation containing 20 mg/kg carbamazepine solubilized in the microemulsion of the preset invention were higher than those measured for the commercial formulation Tegretol. Thus the efficiency of delivering carbamazepine solubilized in the microemulsions of the present invention is higher than that of the commercial Tegretol® suspension.

Analysis of the serum levels of the solubilized (present invention) and suspended (Tegretol®) carbamazepine reveals in FIG. 17 that whereas the suspended carbamazepine is a short term effect, the maximum serum concentration reaches its maximum between 30 to 60 minutes after oral administration, the solubilized form of the present invention has more constant release where the amount in the serum constantly increases such that after 6 hours the concentration continues growing.

1. A microemulsion pharmaceutical composition comprising:
(i) Carbamazepine;
(ii) oil phase;
(iii) at least one C₂-C₅ alcohol as solvent; and
(iv) at least one non ionic surfactant.

2. A microemulsion pharmaceutical composition according to claim 1, comprising 0.1-8 wt % carbamazepine, 10-25 wt % oil phase, 10-25 wt % of the at least one C₂-C₅ alcohol as solvent, and 50-70 wt % of said at least one non ionic surfactant.

3. A microemulsion pharmaceutical composition according to claim 1 further comprising an aqueous phase.

4. A microemulsion pharmaceutical composition according to claim 3 wherein the aqueous phase is selected from water or a mixture of water and C₂-C₅ alcohol as a co-solvent, said aqueous phase being up to 95 wt %.

5. A microemulsion pharmaceutical composition according to claims 1-3 wherein the oil phase is selected from the group consisting of D-limonene, esterified compounds of fatty acids and primary alcohols, propylene glycol mono-C₆₋₁₂ fatty acid esters, glycerol esters of carboxylic acids, medium chain triglycerides having 8 to 20 carbons, or their mixtures.

6. A microemulsion pharmaceutical composition according to claim 4 wherein the C₂-C₅ alcohol is selected from mono hydroxyl alcohols selected from the group consisting of methanol, ethanol, propanol, butanol, pentanol, from bi- or tri-hydroxy alcohols selected from the group consisting of ethylene glycol and propylene glycol or their mixtures.

7. A microemulsion pharmaceutical composition according to claim 1, wherein the non-ionic surfactant is selected from the group consisting of Brij 96, Tween 60, Tween 40 or Tween 80.

8. A microemulsion pharmaceutical composition according to claims 1-3, further comprising an amphiphilic co-surfactant selected from the group consisting of non-ionic surfactants and alcohols, alkanoic acids, alkanediols and alkyl amines or their mixtures.

9. A microemulsion pharmaceutical composition comprising:
(i) Carbamazepine;
(ii) oil phase selected from D-limonene or triacetin;
(iii) at least one C₂-C₅ alcohol as solvent;
(iv) an aqueous phase; and
(v) a non ionic surfactant selected from Brij 96, Tween 60 or Tween 80

10. A pharmaceutical composition according to claim 9 wherein the aqueous phase is water or a mixture of water C₂-C₅ alcohol wherein the C₂-C₅ alcohol is selected from mono hydroxyl alcohols selected from the group consisting of methanol, ethanol, propanol, butanol, pentanol, from bi- or tri-hydroxy alcohols selected from the group consisting of ethylene glycol and propylene glycol or their mixtures.

11. A pharmaceutical composition according to claim 9 or 10, wherein the oil phase is selected from triacetin and vitamin E or D-limonene.

12. A pharmaceutical composition according to claim 9 or 10, wherein the oil phase is selected from triacetin and tocopherol acetate or D-limonene.

13. The composition of claim 1 or 9 wherein the formulation comprises thermodynamically stable molecular species having a diameter of between 10 to 100 nm.

14. The pharmaceutical system of claim 1, wherein the at least one non-ionic surfactant is a non-ionic hydrophilic surfactant having an hydrophile-lipophile-balance (HLB) value greater than or equal to about 10.

15. The pharmaceutical system of claim 14, wherein the non-ionic surfactant is selected from the group consisting of alkylglucosides; alkylmaltosides; alkylthioglucosides; lauryl macrogolglycerides; polyoxyethylene alkyl ethers; polyoxyethylene alkylphenols; polyethylene glycol fatty acids esters; polyethylene glycol glycerol fatty acid esters; polyoxyethylene sorbitan fatty acid esters; polyoxyethylene-polyoxypropylene block copolymers; polyglycerol fatty acid esters; polyoxyethylene glycerides; polyoxyethylene vegetable oils; polyoxyethylene hydrogenated vegetable oils; reaction products of polyols and at least one member of the group consisting of fatty acids, glycerides, vegetable oils, and hydrogenated vegetable oils; sugar esters, sugar ethers; sucroglyccrides; and mixtures thereof.

16. The pharmaceutical system of claim 14, wherein the non-ionic hydrophilic surfactant is selected from the group consisting of polyoxyethylene alkylethers; polyethylene glycol fatty acids esters; polyethylene glycol glycerol fatty acid esters; polyoxyethylene sorbitan fatty acid esters; polyoxyethylene-polyoxypropylene block copolymers; polyglycerol fatty acid esters; polyoxyethylene glycerides; polyoxyethylene vegetable oils; polyoxyethylene hydrogenated vegetable oils; reaction products of polyols and at least one member of the group consisting of fatty acids, glycerides, vegetable oils, and hydrogenated vegetable oils; and mixtures thereof.

17. The pharmaceutical system of claim 16, wherein the non-ionic hydrophilic surfactant is the reaction product of a polyol and a monoglyceride, diglyceride, triglyceride, or a mixture thereof.

18. The pharmaceutical system of claim 17, wherein the reaction product comprises a transesterification product.

19. The pharmaceutical system of claim 17, wherein the polyol is glycerol, ethylene glycol, polyethylene glycol, sorbitol, propylene glycol, pentaerythritol, a saccharide, or a mixture thereof.

20. The pharmaceutical system of claim 18, wherein the hydrophilic surfactant is selected from the group consisting of PEG-10 laurate, PEG-12 laurate, PEG-20 laurate, PEG-32 laurate, PEG-32 dilaurate, PEG-12 oleate, PEG-15 oleate, PEG-20 oleate, PEG-20 dioleate, PEG-32 oleate, PEG-200 oleate, PEG-400 oleate, PEG-15 stearate, PEG-32 distearate, PEG40 stearate, PEG-100 stearate, PEG-20 dilaurate, PEG-32 dioleate, PEG-20 glyceryl laurate, PEG-30 glyceryl laurate, PEG-20 glyceryl stearate, PEG-20 glyceryl oleate, PEG-30 glyceryl oleate, PEG-30 glyceryl laurate, PEG-40 glyceryl laurate, PEG-40 palm kernel oil, PEG-50 hydrogenated castor oil, PEG-40 castor oil, PEG-35 castor oil, PEG-60 castor oil, PEG-40 hydrogenated castor oil, PEG-60 hydrogenated castor oil, PEG-60 corn oil, PEG-6 caprate/caprylate monoglycerides, PEG-6 caprate/caprylate diglycerides, PEG-8 caprate/caprylate monoglycerides, PEG -8 caprate/caprylate diglycerides, polyglyceryl-10 laurate, PEG-40 sorbitan oleate, PEG-80 sorbitan laurate, polysorbate 20, polysorbate 80, POE-9 lauryl ether, POE-23 lauryl ether, POE-10 oleyl ether, POE-20 oleyl ether, POE-20 stearyl ether, tocopheryl PEG-100 succinate, polyglyceryl-10 oleate, Tween 40, Tween 60, sucrose monostearate, sucrose monolaurate, sucrose monopalmitate, PEG 10-100 nonyl phenol series, PEG 15-100 octyl phenol series, a poloxamer, and combinations thereof.

21. The pharmaceutical system of claim 14, wherein the hydrophilic surfactant is selected from the group consisting of PEG-20 laurate, PEG-20 oleate, PEG-35 castor oil, PEG-40 palm kernel oil, PEG-40 hydrogenated castor oil, PEG-60 corn oil, polyglyceryl-10 laurate, PEG-6 caprate/caprylate monoglycerides, PEG-6 caprate/caprylate diglycerides, PEG-8 caprate/caprylate monoglycerides, PEG-8 caprate/caprylate diglycerides; polysorbate 20, polysorbate 80, POE-9 lauryl ether, POE-23 lauryl ether, POE-10 oleyl ether, sucrose monostearate, sucrose monolaurate, a poloxamer, and combinations thereof.

22. The pharmaceutical system of claim 14, wherein the hydrophilic surfactant is selected from the group consisting of PEG-35 castor oil, PEG-40 hydrogenated castor oil, PEG-60 corn oil, PEG-6 caprate/caprylate monoglycerides, PEG-6 caprate/caprylate diglycerides, PEG-8 caprate/caprylate monoglycerides, PEG-8 caprate/caprylate diglycerides, polysorbate 20, polysorbate 60, polysorbate 40, polysorbate 80, tocopheryl PEG-1000 succinate, a poloxamer, and combinations thereof.

23. The pharmaceutical system of claim 1, substantially being free of water.

24. The pharmaceutical system of claim 1 in the form of a preconcentrate in a liquid, or as an aqueous or organic diluted preconcentrate.

25. The pharmaceutical system of claim 1, wherein the dosage form of the composition is a starch capsule, a cellulosic capsule, a hard gelatin capsule or a soft gelatin capsule.

26. The pharmaceutical system of claim 1, wherein the dosage form is formulated for immediate release, controlled release, extended release, delayed release, targeted release, or targeted delayed release.

27. The pharmaceutical system of claim 1, wherein the dosage form of the composition is a solution, spray, gel, drops, syrup or elixir.

28. The pharmaceutical system of claim 1, enhancing two fold the permeability of Carbamazepine in comparison to the available commercial formulation Tegretol.

29. A method of increasing the bioavilability of Carbamazepine comprising administering the composition according to claim 1 or 9.

30. The method of claim 29 wherein the composition is administered orally, topically, rectally, vaginally, parenterally, intramuscularly, intradermally, subcutaneously, intraparitoneally, or intravenously.
